(19) United States
(12) Patent Application Publication
(10) Pub. No.: US 2022/0249235 A1
(43) Pub. Date: Aug. 11, 2022

(54) SLEEVE ELEMENT TO BE PLACED ON A NECK OF A PROSTHETIC HIP OR SHOULDER IMPLANT

(71) Applicants: SENTRYX B.V., Austerlitz (NL); UMC Utrecht Holding B.V., Utrecht (NL)

(72) Inventors: Joannes Jacobus VERLAAN, Zeist (NL); Bas Jeroen OOSTERMAN, Zeist (NL); Floris Rudolf VAN TOL, Austerlitz (NL); Jasper Gerard STEVERINK, Austerlitz (NL)

(21) Appl. No.: 17/613,512

(22) PCT Filed: May 27, 2020
(86) PCT No.: PCT/EP2020/064721

§ 371 Date: Nov. 23, 2021

(30) Foreign Application Priority Data
May 27, 2019 (NL) 2023208

(51) Int. Cl. A61F 2/30

(52) U.S. Cl. CPC A61F 2/30734; A61F 2002/30738; A61F 2002/3625; A61F 2002/3007; A61F 2002/30064; A61F 2002/30677

(57) ABSTRACT

The invention provides a sleeve element (1) to be placed on a neck (53) of a prosthetic hip (51) or shoulder implant, wherein the sleeve element is made of a biodegradable elastically deformable material comprising a medical active agent to be released from the sleeve element. The sleeve element may comprise a longitudinal channel (2) and a longitudinal slit (3) extending over the length of the longitudinal channel and between the longitudinal channel and an outer surface of the sleeve element, wherein the sleeve element is elastically deformable between a closed state and an opened state, wherein a width of the longitudinal slit in the opened state is larger than the width of the longitudinal slit in the closed state. The invention also provides a prosthetic kit, comprising a prosthetic hip or shoulder implant having a neck, and the above sleeve element.

The present invention relates to a sleeve element to be placed on a neck of a prosthetic hip or shoulder implant. The invention further relates to a prosthetic kit comprising a prosthetic hip or shoulder implant and such sleeve element.

Osteoarthritis is seen as one of the most common and incapacitating conditions globally. An effective surgical intervention to alleviate the symptoms caused by hip- or shoulder-osteoarthritis is hip or shoulder joint replacement surgery, wherein a prosthetic hip or shoulder implant is used to replace the affected hip or shoulder. Due to improvements in overall life expectancy and the increasing prevalence of obesity, more and more patients are suffering from osteoarthritis resulting in an increase in the annual number of joint replacements. Over the past two decades, the importance of fast recovery after total joint replacement surgery has gained attention. Numerous studies show that early mobilization leads to a reduction in length of hospital stay, morbidity and overall convalescence without compromising on patient or implant safety.

One of the cornerstones of fast recovery is adequate pain management. In most classic multimodal pain regimens, systemic analgesics such as opioids play a vital role in establishing adequate pain relief. However, there are many systemic side-effects associated with opioid usage which may counteract its analgesic efficacy.

One known alternative for systemic analgesics is Local Infiltration Analgesia (LIA). In LIA, a depot of a local anaesthetic, commonly one of the amide-type, is deposited intraarticularly at the end of the surgical procedure. However, due to a maximum biological half-life of 12 hours for amide-type anaesthetics, the effects of LIA on early mobilization are short-lived. This may result in a decrease in convalescence and an increase in length of hospital stay and morbidity.

To enable extended local pain relief in joint replacement surgery, Continuous Wound Infusion (CWI) techniques have been developed. In this method, a catheter is placed intraarticularly at the end of the surgical procedure which is then used for continuous infusion of a local anaesthetic. Even though this may provide adequate pain relief over an extended period of time, the presence of the catheter may provide an entrance for bacteria which can lead to an increased infection rate. Furthermore, as long as the catheter is in place the patient cannot be discharged home.

Prior art has been published which proposes the integration of carrier devices into hip or knee prostheses, which can then be filled with pharmacons such as local anaesthetics.

For example, US 2011/0015754 A1 discloses an articular implant comprising at least two cavities. The articular implant comprises a femoral or humeral component suited to be fixed to a support bone. The femoral or humeral component comprises a neck configured to support a head suited to being arranged in an articular area. The two cavities are provided with leak tight walls formed at the head made of a thermoplastic material. Said cavities emerging at the surface of the device in contact with the implant site, and means for separating cavities from the implant site, enabling the diffusion of a liquid on either side of said separating means. The separating means are caps that are provided to close the cavities, wherein openings are provided in the caps to enable diffusion of the liquid to the implant site.

The liquid diffused from the cavities may comprise two active substances, that may be chosen among the group of antibiotics, antiseptics, anti-inflammatories, analgesics, antimitotics, anaesthetics or a mixture thereof.

A drawback of the implant of US 2011/0015754 A1 is that a specific hip or shoulder implant has to be provided, in order to provide a local pain treatment after implantation of the hip or shoulder implant. In practice, a relatively long time is needed to introduce a new type of prosthesis on the market. A follow-up of at least 10 years is needed to prove the efficacy, safety and durability of the prosthesis.

US 2009/130167 A1 discloses an implantable drug delivery device. In an embodiment, the drug delivery device is adapted for use with a hip replacement prosthesis. The drug delivery device comprises a body to be arranged on the stem of the hip prosthesis. The body comprises in its outer surface reservoirs to store a volume of drugs. The inner surface of the device circumscribing the aperture may include a plastic material that is relatively soft in comparison to the rest of the body. The plastic material may form an inner liner that deforms, such as when the aperture receives the hip stem, to assist in securing the device bout the hip stem.

US 2003/191537 A1 discloses a constraining ring for use with a hip replacement prosthesis. The ring is arranged to be mounted on the acetabular cup of the prosthesis to maintain a ball of the femoral component in the acetabular cu. The ring may be made of a biologically reabsorbable material.

It is an aim of the present invention to provide a device and/or method that provides an alternative way of delivering a medical active agent after implantation of a prosthetic hip or shoulder implant, for example for pain treatment after implantation of a prosthetic hip or shoulder implant.

The invention provides a sleeve element to be placed on a neck of a prosthetic hip or shoulder implant,

wherein the sleeve element is made of a biodegradable, elastically deformable material comprising one or more medical active agents.

The advantage of the sleeve element of the invention is that the sleeve element may be arranged on the neck of a prosthetic hip or shoulder implant during a surgical hip or shoulder replacement procedure without the need of using a separately designed shoulder or hip implant. The sleeve element may be designed to release the medical active agent during a predetermined period of at least 48 hours, for example at least 72 hours.

The sleeve element of the invention is aimed at providing local pain relief for a sustained period to allow for early mobilization of patients undergoing (total) joint replacement surgery.

The sleeve element may be provided as a universal element that can be mounted on a wide variety of implants. The femoral components presently used as part of a prosthetic hip implant have a shaft part, the so-called neck, on which a femoral head is mounted. It has been found that these shaft parts or necks have substantially the same dimensions for various implants of different manufacturers. The use of a single sleeve element that can be arranged on the neck of the femoral component of these various implants facilitates the local release of a pain treatment medicament or other medical active agent without the need to substantially adapt the surgical procedure and/or the implants presently used.

The sleeve element may, for example, be placed on the neck of the femoral component after the hip prosthesis is implanted, just before the wound is closed. Furthermore, the sleeve may be placed on the neck of previously implanted prosthesis in the case of a reoperation to said prosthesis, for example in the case of surgical debridement due to an infection to the prosthesis. Alternatively, the sleeve element may be placed on the neck just before the head is mounted on the neck of the femoral component.

The term “medical active agent”, as used herein, refers to any substance used internally as a medicine/drug for the treatment, cure, or prevention of a disease or disorder. Examples of pharmaceutical or biological compounds that can be administered as a medical active agent using the sleeve element of the present invention are for example anaesthetics, non-steroidal anti-inflammatory drugs (NSAID's), antibiotics, bone growth stimulating agents, analgesics, chemotherapeutic agents, steroids (including retinoids), hormones, antimicrobials, antivirals, anti-inflammatory compounds, radiation absorbers, including UV-absorbers, vaccines, and stem cells.

The medical active agent is included in the composition in an amount sufficient to deliver to the host patient an effective amount to achieve a desired effect. The amount of medical active agent incorporated into the composition depends upon the desired release profile, the concentration of agent required for a biological effect, and the desired period of release for the agent.

The sleeve element of the invention may incorporate one or more medical active agents, for example a first medical active agent for pain treatment and a second antibiotic medical active agent to treat or prevent bacterial infections.

The medical active agent may be substantially equally distributed within the volume of the elastically deformable material. This may result in a relatively low concentration of medical active agent in the sleeve element, while at the same time providing a relatively high total quantity of the medical active agent.

The shape and volume of the sleeve element may be selected to make maximal use of the intra articular space without hindering the range of motion of the components of the prosthetic hip or shoulder implant. A relatively large volume of the sleeve element allows to provide a relatively large amount of medical active agent with a relatively low concentration of the medical active agent.

The sleeve element is made of elastically deformable material. The deformable material may facilitate the arrangement of the sleeve element on the neck of the prosthetic hip or shoulder implant and facilitate the adaptation of the shape of the sleeve element to the shape of the neck. The deformable material may allow the sleeve element to be applied to a further range of different sized and/or shaped necks of prosthetic hip or shoulder implants. The deformation of the material may also be advantageous when the sleeve element is, for example inadvertently, within the range of motion of the components of the prosthetic hip or shoulder implant. Due to the available elastic deformation, the deformable material may be deformed and therewith not directly hinder the relative movement of these components, when one of the components comes into contact with the sleeve element.

For example, a hip prosthesis comprises a femoral component and an acetabular cup. The femoral component comprises the neck on which a ball shaped femoral head is placed. After implantation, the femoral component may articulate with respect to the acetabular cup. Since the sleeve element is provided on the neck of the femoral component, the sleeve element will be close to or could even come into contact with the components of the prosthetic implant.

The main body of the device of US 2009/130167 A1 is a rigid element. This rigid element must have relatively small dimensions to prevent that the element may interfere with the movements of the prosthetic implant, for example that it becomes pinched between the acetabular cup and the femoral component, since such interference may lead to discomfort for the patient, damage or wear of the prosthetic implant and/or sleeve element and even to dislocation of the prosthetic implant.

Furthermore, the rigid element of US 2009/130167 A1 has to be fixated with bone cement on the neck of the prosthetic implant to ensure that the rigid element will not displace with respect to the neck to a position in which the rigid element may interfere with the relative movements of the components of the prosthetic implant.

Moreover, the device of US 2009/130167 A1 is designed for a specific prosthetic implant, whereby the dimensions of the inner surface of the sleeve element are adjusted to the outer dimensions of the neck of this specific prosthetic implant. Since there are many different types of prosthetic implants available with usually different sizes for each type, many different sizes and shapes of the sleeve element of US 2009/130167 A1 are required for these different prosthetic implants.

By making the sleeve element of elastically deformable material, the whole sleeve element becomes a flexible and/or soft object having a shape that may easily adapt, when needed, to the prosthetic implant. This means that the available elastic deformation of the sleeve element at its inner side may facilitate the arrangement of the sleeve element on the neck of the prosthetic implant. This flexibility is for example advantageous to arrange one embodiment of a sleeve element on differently sized and shaped necks of prosthetic implants, i.e. without the need to provide a separately designed sleeve elements for differently sized and shaped necks.

The available elastic deformation at the outer side of the sleeve element may prevent that the movement of the prosthetic implant is obstructed by the presence of the sleeve element. The advantage of this sleeve element is that, when desired, the sleeve element may be provided with relatively large dimensions, as interference of the sleeve element with the movements of the prosthetic implant may at least partly be compensated by elastic deformation of the sleeve element. The advantage of a larger sleeve element is that the medical active agents may be distributed more evenly within the material of the prosthetic implant resulting in a lower maximum concentration level of medical active agent in a respective volume of the material of the sleeve element. This may be beneficial for reducing the risk of toxicity of the sleeve element and also allows to adjust more precisely the release profile of the medical active agent.

Also, for some medical active agents it may be technically challenging to provide the medical active agent with a relatively high concentration in a carrier. For instance, it may be difficult to obtain a relatively high concentration of an hydrophobic drug, such as bupivacaine, in a hydrogel. For bupivacaine, for example, it may be challenging to obtain a concentration of at least 5% in hydrogel. For other hydrophobic drugs, this percentage may be even lower.

By providing a larger volume of the sleeve element, more medical active agent can be arranged within the sleeve element without the need to increase the concentration of the medical active agent in the sleeve element.

The relatively large volume of the sleeve element may further be beneficial to include multiple medical active agents in the sleeve element with the additional advantage that the release of the multiple medical active agents can be controlled more accurately.

In the event of a contact between the sleeve element and for example the acetabular cup or surrounding tissue, the acetabular cup and/or the sleeve element will be less susceptible to abrasive forces that may lead to damage/wear of the components of the prosthetic implant, surrounding tissue and/or the sleeve element, when compared to the rigid sleeve element of US 2009/130167 A1.

Moreover, since the sleeve element will, when needed, adapt its shape to the components of the prosthetic implant, the sleeve element does not have to be fixated to a predetermined specific position on the neck of the prosthetic implant. Even when there is a displacement of the sleeve element with respect to the neck of the prosthetic implant, the softness/flexibility of the sleeve element allows the sleeve element to adapt its shape to the components between which the sleeve element is pinched.

In an embodiment, the elastic modulus of the biodegradable, elastically deformable material is between 1 kPa and 1 MPa. An elastic modulus (Young's modulus, modus of elasticity) in this range may provide a sleeve element having the flexibility/softness desired to obtain the above-mentioned properties of the sleeve element.

In an embodiment, the sleeve element is completely degraded within 12 months, for example within 1 to 9 months, such as within 1 to 6 months. This relatively short degradation time has the advantage that the sleeve element is only present on the neck of the prosthetic implant for a limited time.

In an embodiment, the biodegradable, elastically deformable material is a polymer material. A polymer material is a suitable material to create a sleeve element which allows elastic deformation that provides the desired level of flexibility/softness.

In an embodiment, the sleeve element is free of fixation elements to fixate the sleeve element to the neck of the prosthesis. Since the sleeve element is elastically deformable, it may adapt its shape when it becomes pinched as a result of movements of the prosthetic implant. Therefore, the need to fixate the sleeve element to the neck is substantially smaller. The advantage of a sleeve element free of fixation elements is that the sleeve element can be arranged in a short time on the neck of the prosthetic implant. This is beneficial since it is desirable that the arrangement of the sleeve element does not have a large impact on the (duration of) standard procedures for placement of a prosthetic hip or shoulder implant. Also, when needed, for example during a reoperation of the prosthetic hip or shoulder implant, the position of the sleeve element can be more easily adapted and/or the sleeve element can be more easily removed from the neck of the prosthetic implant, and possibly replaced by a new sleeve element.

In an embodiment, the sleeve element has a compressive strain of at least 5% at the elastic limit of the elastically deformable material in a direction in which a compressive force is exerted on the elastically deformable material, for example at least 15%, such as at least 30%. The compressive strain is calculated as a change in dimension in a compression direction in which a compressive force is exerted on the sleeve element divided by the original dimension of the sleeve element in the compression direction.

This means that a dimension of the sleeve element in the compression direction in which a compressive force is exerted on the sleeve element may be reduced to at least below 95%, for example at least below 85%, such as at least below 70% of its uncompressed dimension without resulting in plastic deformation of the sleeve element.

In an embodiment, the compressive strain of the sleeve element may be in the range of 10% to 60%, for example 15% to 50% at the elastic limit of the elastically deformable material in a direction in which a compressive force is exerted on the elastically deformable material.

In an embodiment, the sleeve element is designed for one or more specific types prosthetic hip or shoulder implants, and/or provided as a universal sleeve element for prosthetic hip or shoulder implants.

The sleeve element may be used for human and/or animal prosthetic hip or shoulder implants.

The sleeve element may comprise a longitudinal channel through which the neck of the prosthetic hip or shoulder implant may extend after placement of the sleeve element on the neck. The longitudinal channel may extend from one side of the sleeve element to another, opposite side of the sleeve element. The inner dimensions of the longitudinal channel may vary over the length of the longitudinal channel. For example, the inner dimensions of the longitudinal channel may taper outwards from one side towards the other side of the sleeve element. The inner dimensions of the longitudinal channel may be adapted to accommodate a large variety of prosthetic hip and/or shoulder implants.

In an embodiment, the biodegradable elastically deformable material comprises a main body and a coating coated on at least part of a surface of the main body, wherein the medical active agent is comprised in the main body, wherein the coating comprises a coating material and the main body comprises a main body material, wherein the coating material is stiffer than the main body material.

This construction further allows to create a sleeve element with a desired release profile of the medical active agent. The coating may be porous or be provided with openings to allow the medical active agent to diffuse from the main body to the environment of the sleeve element, i.e. the location of the newly implanted hip or shoulder prosthesis.

The coating may be provided on the whole surface of the main body, or only on a part thereof.

In an embodiment, the sleeve element comprises a longitudinal channel and a longitudinal slit extending over the length of the longitudinal channel and between the longitudinal channel and an outer surface of the sleeve element, wherein the sleeve element is elastically deformable between a closed state and an opened state, wherein a width of the longitudinal slit in the opened state is larger than the width of the longitudinal slit in the closed state.

The sleeve element may be elastically deformable between a closed state and an opened state. The closed state is intended to hold the sleeve element on the neck of the prosthetic hip or shoulder implant, and the opened state is intended to be used to place the sleeve element on or remove the sleeve element from the neck of the prosthetic hip or shoulder implant. In the opened state the width of the longitudinal slit is sufficiently large to place the sleeve element over the neck. This means that the width of the slit is at least of the same dimensions as the cross dimension of the neck. In the closed state, the width of the longitudinal slit is smaller than the cross dimension of the neck so that the sleeve element will not easily be displaced from the neck.

In the closed state, the sleeve element may be properly positioned on the neck, for example the neck of a femoral component of the hip implant. The longitudinal channel may advantageously comprise an inner surface that substantially corresponds with an outer surface of the neck of the prosthetic hip or shoulder implant. This allows the sleeve element to be arranged on a single predetermined position on the neck.

In the opened state, the width of the longitudinal slit allows to place the sleeve element on the neck. This can be done at the end of the surgical implant procedure. Thus, only after it has been concluded that the hip or shoulder implant has been successfully implanted into a human or animal body, the sleeve element may be arranged on the neck of the hip or shoulder implant. The placement of the sleeve element therefore does not have a large impact on the presently used surgical procedure. Conveniently, at the end of the surgical procedure, the sleeve element can be arranged on the respective neck thereby minimizing disturbance of the surgical flow.

The width of the longitudinal slit in the closed state may be zero, i.e. the sleeve element completely surrounds the neck, but also larger than zero.

In an embodiment, the sleeve element may be configured to be adhered to the neck. For example, the inner surface forming the longitudinal channel may be provided with an adhesive layer to adhere the sleeve element to the neck.

In an embodiment, the sleeve element in the closed state is substantially non-deformed and in the opened state the sleeve element is elastically deformed. It is of importance that the sleeve element will remain properly positioned on the neck after implantation. If the sleeve element may move from its intended position, the sleeve element may hinder the range of movement of the implanted hip or shoulder, and even cause the hip or shoulder prosthesis to dislocate.

For this reason, it may be advantageous that the mechanical properties are selected such that upon impingement of the sleeve element in between two articulating components of the implanted joint, for example between the femoral component and the acetabular component of a hip prosthesis, the sleeve element may be affected, for example substantially deform or break, before this impingement leads to displacement of the hip or shoulder prosthesis. The sleeve element is made of elastically deformable material to allow some impingement of the sleeve element in between two articulating components of the implanted joint.

When the sleeve element is substantially non-deformed in the closed state, the sleeve element will have a tendency to return to this closed state. This may substantially decrease the chance that the sleeve element will move out of its intended position on the neck.

In an embodiment, the sleeve element comprises a closure device to hold the sleeve element in the closed state. To ensure that the sleeve element will remain properly positioned on the neck after implantation, the sleeve element may comprise a closure device. This closure device may be any device that holds the sleeve element in the closed state. The closure device may prevent that the sleeve element will be pushed in the opened state in which it may be displaced with respect to the neck. Any suitable closure device may be used.

In an embodiment, the longitudinal slit is formed between a first longitudinal edge of the sleeve element and a second longitudinal edge of the sleeve element, wherein the closure device comprises a snap-fit connection between the first longitudinal edge and the second longitudinal edge. The first longitudinal edge and the second longitudinal edge of the sleeve element may be formed to form a snap-fit connection in the closed state. For example, the first longitudinal edge may comprise one or more bulges that can be snapped in one or more recesses provided in the second longitudinal edge of the sleeve element.

In an embodiment, the closure device may be configured to hold the sleeve element, in the closed state, in two or more positions allowing the internal dimensions of the longitudinal channel, or at least part thereof at the location of the closure device to be adapted to the external dimensions of the neck on which the sleeve element is placed. This will allow the sleeve element to be placed tightly on the neck of different types of hip or shoulder prostheses, even when there are some differences in the external dimensions of the neck in the different types of hip or shoulder prostheses.

In an alternative embodiment, the closure device comprises a closure element that can be arranged around the sleeve element. The separate element may be a band, a strip, a cover or a shell that prevents that the sleeve element may extend from the closed state to the opened state. The closure element may be a separate element or an element connected to the sleeve element. The closure element may be made of a biodegradable material.

In an embodiment, the sleeve element comprises stiffening elements. By providing stiffening elements the stiffness of the sleeve element may be increased. The stiffening elements may for example be arranged to bias the sleeve element to the closed state. By increasing the stiffness of the sleeve element, the risk that the sleeve element is displaced on or from the neck may also be reduced. The stiffening elements may be arranged in or on the sleeve element. The stiffening elements may also be made of a biodegradable material.

In an embodiment, the stiffening elements comprise one or more elastically deformable ribs arranged on or in sleeve element. The elastically deformable ribs may extend substantially tangentially with respect to a longitudinal axis of the sleeve element.

In another embodiment, the one or more stiffening elements may comprise a relatively stiff partially or complete cylindrical element that is arranged in the longitudinal channel and is configured to be mounted on the neck of the hip or shoulder prosthesis.

In an embodiment, the sleeve element comprises a longitudinal channel defined by the sleeve element, wherein a cross section of the sleeve element around the longitudinal channel is continuous. Such sleeve element may only be arranged on the neck when the head is not mounted on the neck. This requires that the sleeve element is already arranged on the neck during the surgical procedure before the head is mounted on the neck, or the head has to be temporarily removed from the neck to place the sleeve element on the neck. The cross section of the sleeve element may have an annular shape. The inner and outer dimensions of the sleeve element, for example the inner and outer diameters, may change in longitudinal direction of the sleeve element. For instance, the diameter of the outer surface of the sleeve element and the inner diameter of the longitudinal channel may taper outwards.

In an embodiment, the main body material comprises a visco-elastic material, a degradable felt material, a sponge-like material, a gelatin, a gel, in particular a hydrogel, a polymer or any combination thereof. These materials are suitable materials to provide a suitable elasticity for the placement of the sleeve element on a neck, by elastically deforming the sleeve element between the closed state and the opened state.

In an embodiment, the sleeve element comprises a frusto-conical outer surface. It is advantageous that the volume of the sleeve element is large, where possible, to hold a relatively large amount of medical active agent with a relative low concentration, that can be gradually released from the sleeve element. At the same time the presence of the sleeve element should not hinder the range of motion of the hip or shoulder implant after implantation as to prevent impingement. By providing a frusto-conical outer shape of the sleeve element, the volume of the sleeve element can be increased thereby more optimally using the intra articular space without hindering movement of the hip or shoulder implant after implantation.

In an embodiment, the sleeve element may comprise one or more markers for radiographic visualization of the sleeve element after implantation, for example radiopaque elements. The markers allow the sleeve element to be detected by radiographic visualization techniques, such as X-ray based imaging techniques.

In an embodiment, indicators such as colour codes may be used to indicate different types of sleeve element. The different types may for example relate to different shapes of the sleeve element, different medical active agents comprised in the sleeve element, or different release profiles, for example quantity and/or release rate provided by the sleeve element.

In an embodiment, the medical active agent is an analgesic and/or an anaesthetic. An anaesthetic can advantageously be used for pain treatment after implantation of a prosthetic hip or shoulder implant. In addition, or alternatively, any other medical active agent may be provided in the sleeve element.

The term “medical active agent”, as used herein, refers to any substance used internally as a medicine/drug for the treatment, cure, or prevention of a disease or disorder. Examples of medical active agent that can be administered using the sleeve element of the present invention are for example anaesthetics, non-steroidal anti-inflammatory drugs (NSAID's), antibiotics, bone growth stimulating agents, analgesics, chemotherapeutic agents, steroids (including retinoids), hormones, antimicrobials, antivirals, anti-inflammatory compounds, radiation absorbers, including UV-absorbers, vaccines, and stem cells.

The medical active agent is included in the sleeve element in an amount sufficient to deliver to the host patient an effective amount to achieve a desired effect. The amount of medical active agent incorporated into the sleeve element depends upon the desired release profile, the concentration of medical active agent required for a biological effect, and the desired period of release of the medical active agent.

In an embodiment, the sleeve element comprises two or more compartments, wherein each compartment comprises one or more medical active agents.

The invention further relates to a prosthetic kit, comprising:

a prosthetic hip or shoulder implant having a neck, and

a sleeve element according to any of the preceding claims.

In an embodiment, an inner surface of the longitudinal channel substantially mates with an outer surface of a part of the neck on which the sleeve element is placed or will be placed.

In an embodiment, the sleeve element is designed such that it can be arranged, in the closed state, in only one position on the neck of the prosthetic hip or shoulder implant.

In an embodiment, the neck is part of a femoral component of a prosthetic hip implant, the femoral component having a neck to receive a femoral head, wherein the sleeve element is designed to be placed on the neck of the femoral component.

In an embodiment, the prosthetic hip implant further comprises an acetabular component, wherein the femoral component and acetabular component are designed to facilitate, after implantation, a range of movement of the femoral component with respect to the acetabular component, wherein outer dimensions of the sleeve element are selected to prevent that relative movement of the femoral component with respect to the acetabular component is blocked by the presence of the sleeve element on the neck of the femoral component.

In an embodiment, the prosthetic kit further comprises a loader arranged to load a sleeve element on the neck of the prosthetic hip or shoulder implant. The loader may comprise a loading mechanism to load the sleeve element on the neck.

The loader may for instance be configured to load a sleeve element having a longitudinal slit on the neck. Such loader may comprise a body, a first jaw element and a second jaw element, wherein the first jaw element and the second jaw element are movable with respect to the main body between a normal position and a loading position. In the normal position, the sleeve element may be arranged in the closed state on the loader. When the first jaw element and the second jaw element are moved from the normal position to the loading position, the sleeve element may be forced from the closed state to the opened state allowing the sleeve element to be placed on a neck of a hip or shoulder prosthesis.

To move the first jaw element and the second jaw element from the normal position to the loading position, the first jaw element and the second jaw element may be provided with guide surfaces that, when pushed against the neck, move the first jaw element and the second jaw element towards the loading position.

In an alternative embodiment, the loader comprises an actuation mechanism to actuate movement of the sleeve element mounted on the loader between the closed state to the opened state, for example by movement of jaw elements on which the sleeve element is arranged.

The invention also provides a method of treatment, for example pain treatment, in joint replacement surgery comprising the steps of:

implanting a prosthetic hip or shoulder implant having a neck into a human or animal body; and

placing a sleeve element as claimed in any of the claims 1-15 on the neck of the prosthetic hip or shoulder implant. The sleeve element may be placed before, during or after actual implantation of the prosthetic hip or shoulder implant on the neck of the implant.

In an embodiment, the sleeve element comprises a longitudinal channel and a longitudinal slit extending over the length of the longitudinal channel and between the longitudinal channel and an outer surface of the sleeve element, wherein the sleeve element is elastically deformable between a closed state and an opened state, wherein a width of the longitudinal slit in the opened state is larger than the width of the longitudinal slit in the closed state, and wherein the sleeve element is held in the opened state while placing the sleeve element on the neck.

Embodiments of the invention will now be described, by way of example only, with reference to the accompanying schematic drawings in which corresponding reference symbols indicate corresponding parts, and in which:

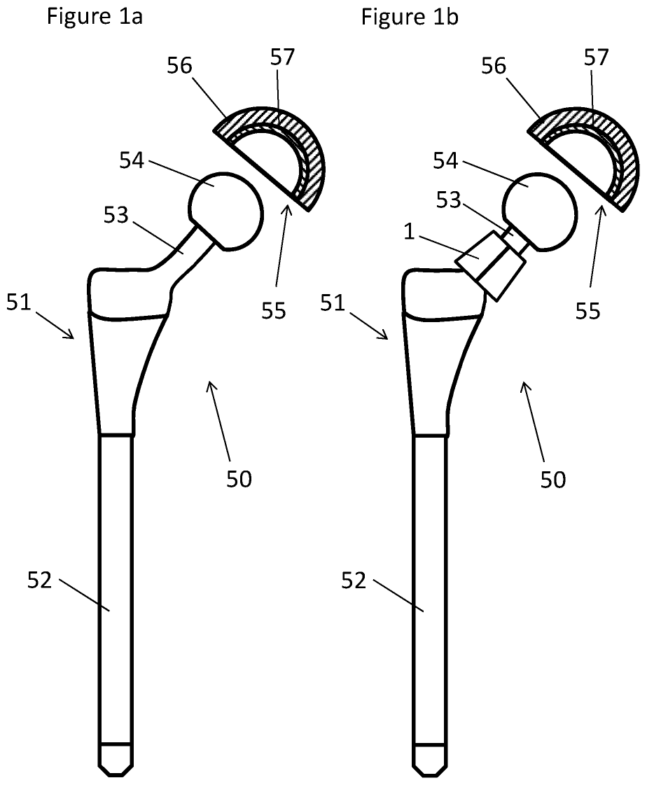

FIG. 1a shows an embodiment of a prosthetic hip implant;

FIG. 1b shows the embodiment of the prosthetic hip implant of FIG. 1a including a first embodiment of a sleeve element arranged on the neck of the femoral component of the prosthetic hip implant;

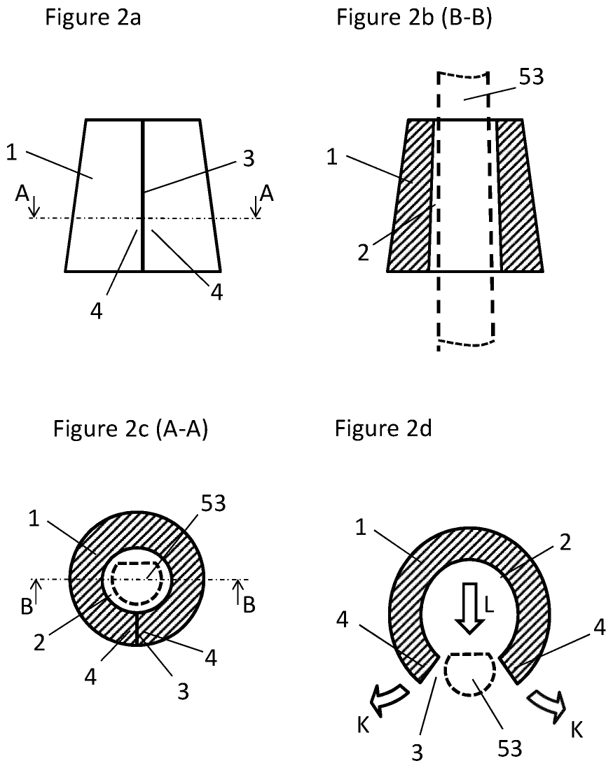

FIG. 2a shows a side view of the first embodiment of a sleeve element;

FIG. 2b shows a longitudinal sectional view B-B of the first embodiment of FIG. 1a;

FIG. 2c shows a cross sectional view A-A of the embodiment of FIG. 1a in closed state;

FIG. 2d shows a cross sectional view of the embodiment of FIG. 1a in opened state;

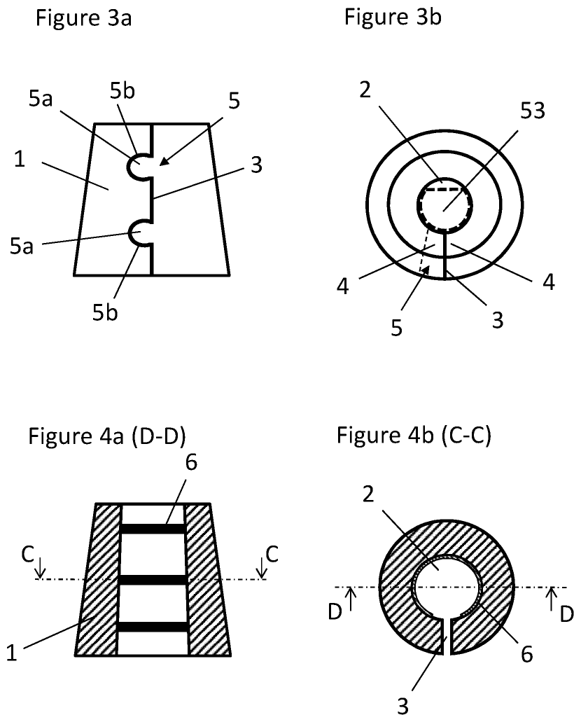

FIG. 3a shows a side view of a second embodiment of a sleeve element;

FIG. 3b shows a top view of the second embodiment of FIG. 3a;

FIG. 4a shows a longitudinal sectional view D-D of a third embodiment of a sleeve element;

FIG. 4b shows a cross sectional view C-C of the embodiment of FIG. 4a;

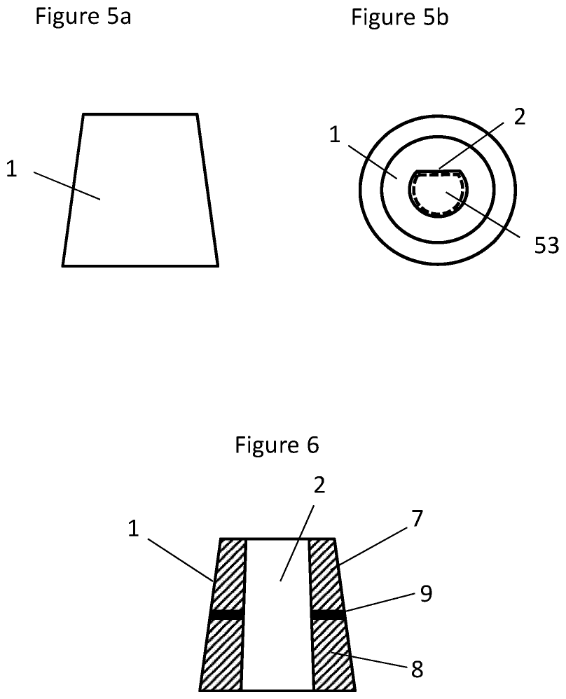

FIG. 5a shows a side view of a fourth embodiment of a sleeve element;

FIG. 5b shows a top view of the fourth embodiment of FIG. 5a;

FIG. 6 shows a longitudinal sectional view of a fifth embodiment of a sleeve element;

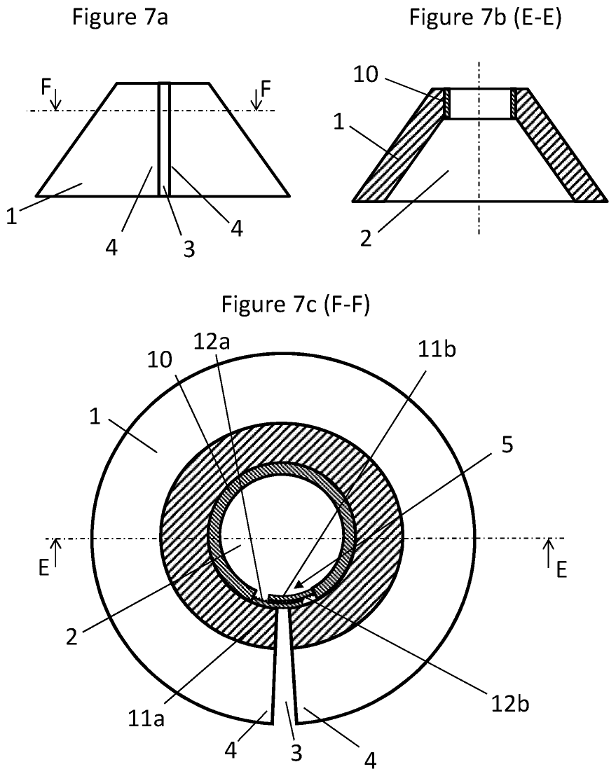

FIG. 7a shows a side view of a sixth embodiment of a sleeve element;

FIG. 7b shows a longitudinal sectional view E-E of the embodiment of FIG. 7a;

FIG. 7c shows a cross sectional view F-F of the embodiment of FIG. 7a;

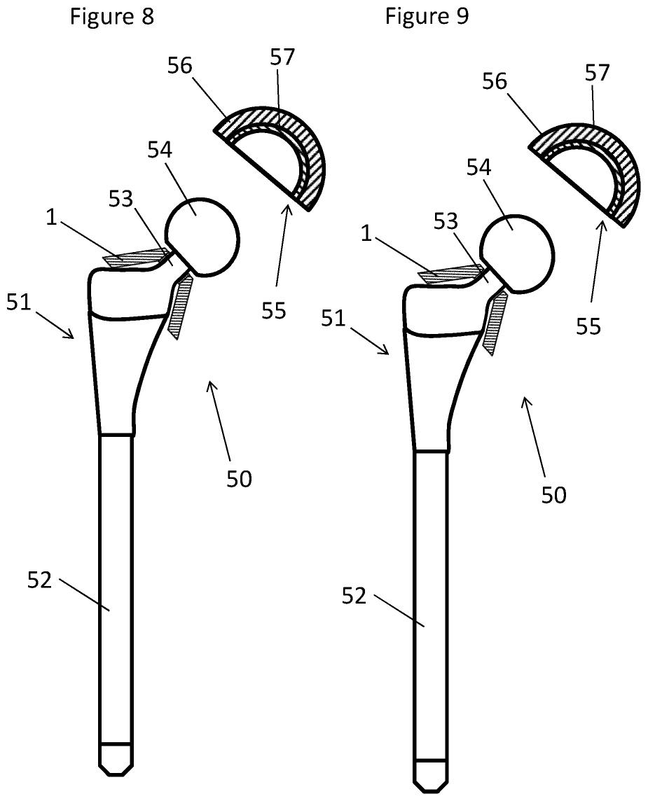

FIG. 8 shows the embodiment of FIG. 7a mounted on a prosthetic hip implant;

FIG. 9 shows a seventh embodiment of a sleeve element mounted on a prosthetic hip implant;

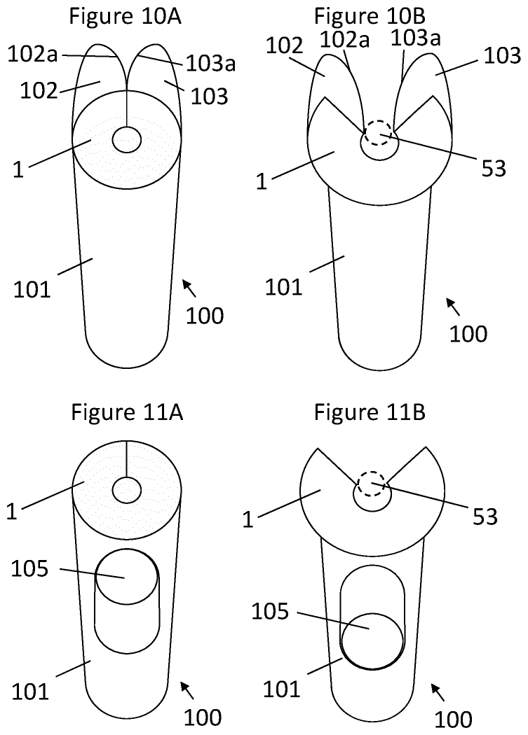

FIGS. 10a and 10b show schematically a first embodiment of a loader configured to arranged the sleeve element on a neck of a prosthetic hip or shoulder implant; and

FIGS. 11a and 11b show schematically a second embodiment of a loader configured to arranged the sleeve element on a neck of a prosthetic hip or shoulder implant.

FIG. 1a shows a prosthetic hip implant generally denoted by reference numeral 50. The prosthetic hip implant 50 comprises a femoral component 51 to be connected to the femur of a patient and a acetabular component 55 to be connected to the acetabulum (hip socket) of a patient.

The femoral component 51 comprises a stem 52 to be placed into the femur, a neck 53 and a head 54 supported on the neck 53. The acetabular component 55 comprises a acetabular cup 56 that may be provided with a liner 57 arranged in the acetabular cup 56.

Prosthetic hip implants 50 are widely known in the art. Hip replacements using these types of prosthetic hip implants 50 is currently one of the most common orthopedic operations, though patient satisfaction short- and long-term varies widely. In view of the possible negative effects of a hip replacement, such as pain and infection risks, there is a need to administer one or more medical active agents to the patient after implantation of the prosthetic hip implant 50.

FIG. 1b shows the prosthetic hip implant 50 of FIG. 1 with a sleeve element 1 according to an embodiment of the invention arranged on the neck 53 of the femoral component 51. The sleeve element 1 is made of a biodegradable elastically deformable material comprising a medical active agent, for example a pain treatment drug, such as an anaesthetic or analgesic.

The sleeve element 1 is configured to release the medical active agent according to a predetermined release profile.

The sleeve element 1 is arranged to be completely degraded within 12 months, for example within 1 to 9 months. This relatively short degradation time has the advantage that the sleeve element 1 is only present on the neck of the prosthetic implant for a limited time.

FIGS. 2a, 2b, 2c and 2d show the embodiment of the sleeve element 1 as depicted in FIG. 1b in more detail.

The sleeve element 1 comprises a longitudinal channel 2 and a longitudinal slit 3. The longitudinal channel 2 is configured to receive the neck 53. In the embodiment shown in FIGS. 2a-2d, the inner dimensions of the longitudinal channel 2 are slightly larger than the outer dimensions of the neck 53 resulting in some play between the sleeve element 1 and the neck 53. In other embodiments, the inner dimensions of the longitudinal channel 2 may be substantially the same as the outer dimensions of the neck 53 resulting in a tight fit between the sleeve element 1 and the neck 53. In yet another embodiment, the inner dimensions of the longitudinal channel 2 may be smaller than the outer dimensions of the neck 53 resulting in some clamping of the sleeve element 1 on the neck 53.

The longitudinal slit 3 extends over the length of the longitudinal channel 2 between the longitudinal channel 2 and an outer surface of the sleeve element 1. As a result, the sleeve element 1 comprises two longitudinal edges 4 that may be arranged against each other to create a closed ring-shaped cross section as shown in FIG. 2c.

The sleeve element 1 is elastically deformable between a closed state, as shown in FIG. 2c, in which the width of the longitudinal slit 3 is smaller than a cross dimension of the neck 53, as shown in FIG. 2c, and an opened state in which the width of the longitudinal slit is at least the same as the cross dimension of the neck 53. In the closed state, the sleeve element 1 may be non-deformed, i.e. without any external force, the sleeve element 1 will be in the closed state shown in FIG. 2c.

The sleeve element 1 can be brought into the opened state by moving the longitudinal edges 4 away from each other, as shown in FIG. 2d by arrows K, to create a distance between the longitudinal edges 4. When the width of the slit 3, i.e. the distance between the longitudinal edges 4, is at least as large as the cross dimension of the neck 53, the sleeve element 1 can be placed on the neck 53, by movement of the sleeve element 1 in the direction L shown in FIG. 2d. When the longitudinal edges 4 are released, the sleeve element 1 will substantially deform back towards the closed state where it is held on the neck 53.

The advantage of this embodiment of the sleeve element 1 is that the sleeve element 1 can be arranged on the neck 53 even when the head 54 is already mounted on the neck 53. As a result, the sleeve element 1 may be arranged on the neck 53 at any suitable stage of the surgical procedure. For example, only after it has been concluded that the prosthetic hip implant 50 has been successfully implanted into a human or animal body, the sleeve element 1 may be arranged on the neck 53. The placement of the sleeve element 1 therefore does not have a large impact on the presently used surgical procedure for prosthetic hip or shoulder implants.

The inner surface of the sleeve element forming the longitudinal channel 2 may at least partially be provided with an adhesive layer that allows the sleeve element 1 to be adhered to the neck 53. This further reduces the chance that the sleeve element 1 inadvertently is moved from its position on the neck 53.

In another embodiment, the sleeve element is free of fixation elements to fixate the sleeve element 1 to the neck 53 of the prosthetic implant. Since the sleeve element 1 is elastically deformable, it may adapt its shape when it becomes pinched as a result of movements of the prosthetic implant, and therefore some displacement of the sleeve element 1 on the neck 53 may be accepted.

The sleeve element 1 is made of a biodegradable elastically deformable material and comprises a main body and a coating coated on at least a part of the surface the main body. The medical active agent is comprised in the main body. The medical active agent may be distributed substantially equally within the material of the main body. This has the advantage that a relatively large volume of medical active agent with a relatively low maximum concentration level of the medical active agent can be arranged in the main body of the sleeve element 1.

The coating comprises a coating material and the main body comprises a main body material. The coating material is stiffer than the main body material. The main body material may for example comprise a visco-elastic material, a degradable felt material, a sponge-like material, a gelatin, a gel, in particular a hydrogel, a polymer or any combination thereof. The sleeve element 1 may also be provided without a coating, i.e. only a main body of the same biodegradable elastically deformable material.

The elastic modulus of the biodegradable, elastically deformable material may be between 1 kPa and 1 MPa.

The sleeve element shown in FIGS. 2a, 2b, and 2c comprises a frusto-conical outer shape. This shape is selected to make maximally use of the intra articular space without hindering the range of motion of the components of the prosthetic hip implant 50. A relatively large volume of the sleeve element 1 allows to provide a relatively large amount of medical active agent with a relatively low concentration of the medical active agent.

The outer dimensions of the sleeve element are selected such that they do not overpass a virtual line between the outer dimensions of the head mounted on the neck of a prosthesis and the outer dimensions of the base of the neck, thereby ensuring no or little interference with the range of motion of the prosthesis. Some interference may be compensated by the deformability of the material of the sleeve element 1.

To compensate any interference of the sleeve element 1 with the movement of the prosthetic implant 50, the sleeve element 1 may have a compressive strain of at least 5%, for example at least 15%, at the elastic limit of the elastically deformable material in a direction in which a compressive force is exerted on the elastically deformable material. The compressive strain may be calculated as a change in dimension in a compression direction in which a compressive force is exerted on the sleeve element divided by the original dimension of the sleeve element in the compression direction.

FIGS. 3a and 3b show a second embodiment of a sleeve element 1. In this embodiment, a closure device 5 is provided to hold the sleeve element 1 in the closed state. The closure device comprises bulges 5a at one longitudinal edge 4 and corresponding recesses 5b at the opposite longitudinal edge 4 of the sleeve element 1. The bulges 5a and the recesses 5b form a snap-fit connection between the longitudinal edges 4. In the closed state of the sleeve element 1, the bulges 5a may be arranged in the recesses 5b to prevent that the longitudinal edges 4 move away from each other.

In alternative embodiments, other closure devices may be provided to hold the sleeve element 1 in the closed state. These closure devices may be integrated in the material of the sleeve element 1, such as the bulges 5a and recesses 5b, but may also be provided as separate devices that can be used to hold the sleeve element 1 in the closed state. These closure devices may for example comprise closure elements such as bands, sutures, cerclages, strips, shells or covers that can be arranged around the sleeve element to hold the sleeve element 1 in the closed state. The complete sleeve element 1 including the closure device may be made of biodegradable materials.

FIGS. 4a and 4b show a third embodiment of a sleeve element 1. On the inner surface forming the longitudinal channel 3 elastically deformable ribs 6 are arranged. The elastically deformable ribs 6 are relatively stiff with respect to the material of the main body of the sleeve element 1. The elastically deformable ribs 6 form stiffening elements that bias the sleeve element 1 to the closed state. The elastically deformable ribs 6 do however not prevent that the sleeve element 1 may be opened to the opened state to arrange the sleeve element 1 on the neck 53 of the femoral component 51. Thus, the sleeve element 1 can still be mounted on the neck 53 after the head 54 of the hip prosthesis implant 51 has been mounted on the neck 53.

The elastically deformable ribs 6 may be created by a coating material coated on the inner surface of the longitudinal channel 2. The elastically deformable ribs 6 may also be created by any other suitable material. In addition, or as an alternative, elastically deformable ribs 6 may be provided in/by the material of the main body of the sleeve element 1 or on the outer side of the sleeve element. Also, other stiffening elements, such as relatively stiff partially cylindrical elements may be provided to increase the stiffness of the sleeve element 1 in order to bias the sleeve element to the closed state. The complete sleeve element, including the stiffening elements 6 may be made of biodegradable materials.

FIGS. 5a and 5b show a fourth embodiment of a sleeve element 1. In this embodiment, the sleeve element 1 does not comprise a longitudinal slit. The cross section of the sleeve element 1 around the longitudinal channel 2 is continuous. The sleeve element 1 of FIGS. 5a and 5b can only be arranged on the neck 53 when the head 54 is not mounted on the neck 53. This requires that the sleeve element 1 is already arranged on the neck 53 during the surgical procedure before the head 54 is mounted on the neck 53, or the head 54 has to be temporarily removed from the neck 53 to place the sleeve element 1 on the neck.

FIG. 5b shows that the shape and dimensions of the cross-section of the longitudinal channel 2 are selected to substantially correspond to shape and dimensions of the cross-section of the neck 53. This provides a relative tight fit of the sleeve element 1 on the neck 53, whereby the sleeve element 53 can only be mounted in a single position on the neck 53. This single mounting position can be used advantageously to ensure that the sleeve element 1 will only occupy the intended space in the hip or should prosthesis. This may in particular be relevant when the sleeve element 1 is designed to maximally use the intra articular space, while at the same time interference with the range of motion of the hip or should prosthesis after implantation should be substantially avoided.

The adaptation of the shape and dimensions of the cross-section of the longitudinal channel 2 to substantially correspond to shape and dimensions of the cross-section of the neck 53 may also be applied in any other embodiment of a sleeve element 1, such as the sleeve elements of the embodiments shown and described in this patent application.

However, since the sleeve element 1 is made of elastically deformable material, the deformable material may also facilitate the arrangement of the sleeve element 1 on the neck of different types and sizes of the prosthetic hip or shoulder implants and facilitate the adaptation of the shape of the sleeve element 1 to the shape of the neck of the respective prosthetic hip or shoulder implant.

FIG. 6 shows a fifth embodiment of a sleeve element 1. The sleeve element 1 comprises a first compartment 7 and a second compartment 8 in the main body, wherein each of the first compartment 7 and the second compartment 8 comprises a medical active agent. The medical active agent in the first compartment 7 may be the same as the medical active agent in the second compartment 8, but the first compartment 7 and the second compartment 8 may be designed to provide different release profiles for the medical active agent. Alternatively, the medical active agent or combination of medical active agents in the first compartment 7 may be different than the medical active agent or combination of medical active agents in the second compartment 8 and having the same or different medical active agent release profiles.

For example, the first compartment 7 may comprise a medical active agent for pain treatment, such as an analgesic or anaesthetic, and the second compartment 8 may comprise an antibiotic to treat or prevent bacterial infections. These medical active agents may also be provided in a single compartment, but the use of two compartments allows to influence and control the release profiles of the two medical active agents more accurately.

In the embodiment of FIG. 6 the first compartment 7 and the second compartment 8 are separated by a separation wall 9. In an alternative embodiment, the first compartment 7 and the second compartment 8 do not have to be separated by a separation wall 9. The first compartment 7 and the second compartment 8 may for example also be distinguished by their differences in characteristics.

FIGS. 7a, 7b and 7c show a sixth embodiment of a sleeve element 1 to be arranged on the neck 53 of a prosthetic hip or shoulder implant.

FIG. 8 shows this sixth embodiment, in cross section, mounted on the neck of the prosthetic hip implant 50 of FIG. 1a.

The sleeve element 1 comprises a longitudinal slit 3 arranged between the longitudinal edges 4. The sleeve element 1 comprises a longitudinal channel 2 that tapers outwards from one side (upper side in FIG. 7b) to the opposite side (bottom side in FIG. 7b). This shape is selected such that the sleeve element 1 that is mounted on the neck 53 may have a relatively large volume, that is arranged, after implantation as shown in FIG. 8, while at the same time, a relative small length of the neck 53 is needed for mounting the sleeve element 1 on the neck 53. At the same time a relatively large amount of medical active agent in a relatively low concentration may be provided near the prosthetic hip implant 50.

In the longitudinal channel 2, the sleeve element 1 comprises a relatively stiff partially cylindrical element 10. This partially cylindrical element 10 acts as a stiffening element that improves the positioning of the sleeve element 1 on the neck 53. The partially cylindrical element 10 is also part of a closure device 5. The closure device 5 comprises two extensions 11a, 11b connected at opposite ends of the partially cylindrical element 10. Each of the two extensions 11a, 11b support ratchet teeth 12a, 12b. The ratchet teeth 12a, 12b allow the extensions 11a, 11b to be locked with respect to each other in multiple relative locking positions. Each of the multiple relative locking positions relates to a specific diameter of the partially cylindrical element 10. Thus, the closure device 5 allows the sleeve element 1 to be tightly mounted on the neck 53 of different types of prosthetic hip implants even when the necks of the different types of prosthetic hip implants may have other dimensions within the range of the relative locking positions of the two extensions 11a, 11b. This tight arrangement of the partially cylindrical element 10 on the neck 53 also reduces the risk of displacement of the sleeve element 1 on the neck 53, in particular sliding over the neck 53.

The stiffness of the partially cylindrical element 10 may be selected such that the shape of the partially cylindrical element 10 may at least partially adapt to a non-cylindrical cross section of the neck 53.

All elements of the sleeve element 1 of FIGS. 7a, 7b and 7c may be made of biodegradable materials.

FIG. 9 shows a seventh embodiment of a sleeve element 1, in cross section, mounted on the neck 53 of the prosthetic hip implant 50 of FIG. 1a. The seventh embodiment comprises generally the same shape as the embodiment of FIGS. 7a, 7b and 7c, but does not comprise the partially cylindrical element 10. The sleeve element 1 of FIG. 8 may be provided with a longitudinal slit, such the sleeve element 1 may be mounted on the neck 53 at the end of a surgical procedure, just before the wound is closed. In an alternative embodiment, the sleeve element 1 may be provided with a continuous ring shaped cross section, i.e. without a longitudinal slit. In such case the sleeve element 1 may for example be arranged on the neck 53 before the head 54 is mounted on the neck 53. When needed, the sleeve element 1 may be fixed to the neck 52, for example by gluing or by providing separate closure elements.

FIGS. 10a and 10b show a first embodiment of a loader 100. The loader 100 is arranged to load a sleeve element 1 having a longitudinal slit 3 on the neck 53 of a prosthetic hip or shoulder implant. The loader 100 comprises a body 101, a first jaw element 102 and a second jaw element 103.

The first jaw element 102 and the second jaw element 103 are movable with respect to the main body between a normal position, shown in FIG. 10a and a loading position, shown in FIG. 10b. In the normal position, the sleeve element 1 may be arranged in the closed state on the loader 100. When the first jaw element 102 and the second jaw element 103 are moved from the normal position to the loading position, the sleeve element 1 will be forced from the closed state to the opened state allowing the sleeve element 1 to be placed on a neck 53 of a hip or shoulder prosthesis. The first jaw element 102 and the second jaw element 103 may be biased towards the normal position by a biasing element. This biasing element may also be formed by the sleeve element 1 arranged on the loader 100.

The first jaw element 102 comprises a first guide surface 102a and the second jaw element 103 comprises a second guide surface 103a. By pushing the neck 53 against the first guide surface 102a and the second guide surface 103a, the first jaw element 102 and the second jaw element 103 may be moved from the normal position towards the loading position. No separate actuation mechanism is required to actuate movement of the first jaw element 102 and the second jaw element 103.

FIGS. 11a and 11b show a second embodiment of a loader 100 arranged to load a sleeve element 1 having a longitudinal slit 3 on the neck 53 of a prosthetic hip or shoulder implant. This loader 100 comprises an actuation mechanism 105 to actuate movement of the sleeve element 1 mounted on the loader 100 between the closed state, shown in FIG. 11a to the opened state, shown in FIG. 11b. The actuation mechanism 105 schematically depicted by a slider may actuate movement of a first jaw element and a second jaw element between the normal position and loading position to move the sleeve element 1 between the closed and opened state.

1. A sleeve element to be placed on a neck of a prosthetic hip or shoulder implant,
wherein the sleeve element is made of a biodegradable, elastically deformable material comprising one or more medical active agents.

2. The sleeve element of claim 1, wherein the elastic modulus of the biodegradable, elastically deformable material is between 1 kPa and 1 MPa.

3. The sleeve element of claim 1, wherein the sleeve element is free of fixation elements to fixate the sleeve element to the neck of the prosthesis.

4. The sleeve element of claim 1, wherein the sleeve element has a compressive strain of at least 5% at the elastic limit of the elastically deformable material in a direction in which a compressive force is exerted on the elastically deformable material.

5. The sleeve element of claim 1, wherein the biodegradable elastically deformable material comprises a main body and a coating coated on at least a part of the surface the main body, wherein the medical active agent is comprised in the main body, wherein the coating comprises a coating material and the main body comprises a main body material, wherein the coating material is stiffer than the main body material.

6. The sleeve element of claim 1, wherein the sleeve element comprises a longitudinal channel and a longitudinal slit extending over the length of the longitudinal channel and between the longitudinal channel and an outer surface of the sleeve element, wherein the sleeve element is elastically deformable between a closed state and an opened state, wherein a width of the longitudinal slit in the opened state is larger than the width of the longitudinal slit in the closed state.

7. The sleeve element of claim 6, wherein the sleeve element in the closed state is substantially non-deformed and in the opened state the sleeve element is elastically deformed.

8. The sleeve element of claim 6, wherein the sleeve element comprises a closure device to hold the sleeve element in the closed state.

9. The sleeve element according to claim 6, wherein the longitudinal slit is formed between a first longitudinal edge of the sleeve element and a second longitudinal edge of the sleeve element, wherein the closure device comprises a snap fit connection between the first longitudinal edge and the second longitudinal edge.

10. The sleeve element according to claim 9, wherein the closure device comprises a closure element that can be arranged around the sleeve element.

11. The sleeve element of claim 1, wherein the sleeve element comprises a longitudinal channel defined by a continuous ring shape around the longitudinal channel.

12. The sleeve element of claim 1, wherein the sleeve element comprises one or more stiffening elements that bias the sleeve element to the closed state.

13. The sleeve element of claim 12, wherein the one or more stiffening elements comprise one or more elastically deformable ribs arranged on or in sleeve element.

14. The sleeve element of claim 5, wherein the main body material comprises a visco-elastic material, a degradable felt material, a sponge-like material, a gelatin, a gel, in particular a hydrogel, a polymer material, or any combination thereof.

15. The sleeve element of claim 1, wherein the sleeve element comprises a shape which is designed to not hinder a range of motion of the hip or shoulder implant after implantation, or a frustro-conical outer shape.

16. (canceled)

17. The sleeve element of claim 1, wherein the medical active agent is an analgesic or an anaesthetic.

18. The sleeve element of claim 1, wherein the sleeve element comprises two or more compartments, wherein each compartment comprises one or more medical active agents.

19. A prosthetic kit, comprising:
a prosthetic hip or shoulder implant having a neck, and
a sleeve element according to claim 1.

20. The prosthetic kit of claim 19, wherein an inner surface of the longitudinal channel substantially mates with an outer surface of a part of the neck on which the sleeve element is placed or will be placed.

21. The prosthetic kit of claim 19, wherein the sleeve element is designed such that it can be arranged, in the closed state, in only one position on the neck of the prosthetic hip or shoulder implant.

22. The prosthetic kit of claim 19, wherein the neck is part of a femoral component of a prosthetic hip implant, the femoral component having a neck to receive a femoral head, wherein the sleeve element is designed to be placed on the neck of the femoral component.

23. The prosthetic kit of claim 22, wherein the prosthetic hip implant further comprises an acetabular component, wherein the femoral component and acetabular component are designed to facilitate, after implantation, a range of movement of the femoral component with respect to the acetabular component, wherein outer dimensions of the sleeve element are selected to prevent that relative movement of the femoral component with respect to the acetabular component is blocked by the presence of the sleeve element on the neck of the femoral component.

24. The prosthetic kit of claim 19, wherein the prosthetic kit further comprises a loader arranged to load a sleeve element on the neck of the prosthetic hip or shoulder implant.
